SEQUENCE LISTING (10 sequences)

SEQ ID NO: 1 - DNA, 20 bp, Artificial Sequence
  primer
ggcgttggtggcgggtgttt

SEQ ID NO: 2 - DNA, 22 bp, Artificial Sequence
  primer
agcccatggacaatgcagccac

SEQ ID NO: 3 - DNA, 24 bp, Artificial Sequence
  primer
cacagagcctcgcctttgccgatc

SEQ ID NO: 4 - DNA, 23 bp, Artificial Sequence
  primer
acgagcgcggcgatatcatcatc

SEQ ID NO: 5 - DNA, 2044 bp, Homo sapiens
ctggacgagtccgagcgcgtcacctcctcacgctgcggctgtcgcccgtgtcccgccggcccgttccgtgtcgccccgcagtgctgcggccgccgcggcaccatggctgtgtttgtcgtgctcctggcgttggtggcgggtgttttggggaacgagtttagtatattaaaatcaccagggtctgttgttttccgaaatggaaattggcctataccaggagagcggatcccagacgtggctgcattgtccatgggcttctctgtgaaagaagacctttcttggccaggactcgcagtgggtaacctgtttcatcgtcctcgggctaccgtcatggtgatggtgaagggagtgaacaaactggctctacccccaggcagtgtcatttcgtaccctttggagaatgcagttccttttagtcttgacagtgttgcaaattccattcactccttattttctgaggaaactcctgttgttttgcagttggctcccagtgaggaaagagtgtatatggtagggaaggcaaactcagtgtttgaagacctttcagtcaccttgcgccagctccgtaatcgcctgtttcaagaaaactctgttctcagttcactccccctcaattctctgagtaggaacaatgaagttgacctgctctttctttctgaactgcaagtgctacatgatatttcaagcttgctgtctcgtcataagcatctagccaaggatcattctcctgatttatattcactggagctggcaggtttggatgaaattgggaagcgttatggggaagactctgaacaattcagagatgcttctaagatccttgttgacgctctgcaaaagtttgcagatgacatgtacagtctttatggtgggaatgcagtggtagagttagtcactgtcaagtcatttgacacctccctcattaggaagacaaggactatccttgaggcaaaacaagcgaagaacccagcaagtccctataaccttgcatataagtataattttgaatattccgtggttttcaacatggtactttggataatgatcgccttggccttggctgtgattatcacctcttacaatatttggaacatggatcctggatatgatagcatcatttataggatgacaaaccagaagattcgaatggattgaatgttacctgtgccagaattagaaaagggggttggaaattggctgttttgttaaaatatatcttttagtgtgctttaaagtagatagtatactttacatttataaaaaaaaatcaaattttgttctttattttgtgtgtgcctgtgatgtttttctagagtgaattatagtattgacgtgaatcccactgtggtatagattccataatatgcttgaatattatgatatagccatttaataacattgatttcattctgtttaatgaatttggaaatatgcactgaaagaaatgtaaaacatttagaatagctcgtgttatggaaaaaagtgcactgaatttattagacaaacttacgaatgcttaacttctttacacagcataggtgaaaatcatatttgggctattgtatactatgaacaatttgtaaatgtcttaatttgatgtaaataactctgaaacaagagaaaaggtttttaacttagagtagccctaaaatatggatgtgcttatataatcgcttagttttggaactgtatctgagtaacagaggacagctgttttttaaccctcttctgcaagtttgttgacctacatgggctaatatggatactaaaaatactacattgatctaagaagaaactagccttgtggagtatatagatgcttttcattatacacacaaaaatccctgagggacattttgaggcatgaatataaaacatttttatttcagtaacttttccccctgtgtaagttactatggtttgtggtacaacttcattctatagaatattaagtggaagtgggtgaattctactttttatgttggagtggaccaatgtctatcaagagtgacaaataaagttaatgatgattccaaaaaaaaaa

SEQ ID NO: 6 - protein, 350 aa, Homo sapiens
MAVFVVLLALVAGVLGNEFSILKSPGSVVFRNGNWPIPGERIPDVAALSMGFSVKEDLSWPGLAVGNLFHRPRATVMVMVKGVNKLALPPGSVISYPLENAVPFSLDSVANSIHSLFSEETPVVLQLAPSEERVYMVGKANSVFEDLSVTLRQLRKRLFQENSVLSSLPLNSHSRNNEVDLLFLSELQVLHDISSLLSRHKHLAKDHSPDLYSLELAGLDEIGKRYGEDSEQFRDASKILVDALQKFADDMYSLYGGNAVVELVTVKSFDTSLIRKTRTILEAKQAKNPASPYNLAYKYNFEYSVVFNMVLWIMIALALAVIITSYNIWNMDPGYDSIIYRMTNQKIRMD

SEQ ID NO: 7 - protein, 14 aa, Homo sapiens
HKHLAKDHSPDLYS

SEQ ID NO: 8 - RNA, 25 nt, Artificial Sequence
  siRNA
uauagggacuugcuggguucuucgc

SEQ ID NO: 9 - RNA, 25 nt, Artificial Sequence
  siRNA
gcgaagaacccagcaagucccuaua

SEQ ID NO: 10 - protein, 350 aa, Mus musculus
MAVLVVLLFFLVAGALGNEFSILRSPGSVVFRNGNWPIPGDRIPDVAALSMGFSVKEDLSWPGLAVGNLFHRPRATIMVMVKGVDKLALPAGSVISYPLENAVPFSLDSVANSIHSLFSEETPVVLQLAPSEERVYMVGKANSVFEDLSVTLRQLRNRLFQENSLLNSLPLNSLSRNNEVDLLFLSELQVLHDISSLLSRHKHLAKDHSPDLYSLELAGLDELGKRYGEDSEQFRDASKILVDALQKFADDMYSLYGGNAVVELVTVKSFDTSLVRKSRTILEAKQENTQSPYNLAYKYNLEYSVVFNLVLWIMIGLALAVIITSYNIWNMDPGYDSIIYRMTNQKIRID

(19) United States
(12) Patent Application Publication
(10) Pub. No.: US 2018/0080939 A1
(43) Pub. Date: Mar. 22, 2018

(54) CANCER MARKER AND THE USE THEREOF

(71) Applicant: National University Corporation Kagawa University, Takamatsu-shi (JP)

(72) Inventors: Akira NISHIYAMA, Kagawa (JP); Fumiaki SUZUKI, Gifu (JP)

(21) Appl. No.: 15/821,146

(22) Filed: Nov. 22, 2017

Related U.S. Application Data
(62) Division of application No. 14/427,200, filed on Mar. 10, 2015, now abandoned.

(30) Foreign Application Priority Data
Sep. 11, 2012 (JP) 2012-199508

(51) Int. Cl. G01N 33/574; A61K 39/395; C07K 14/705; C07K 16/28; A61K 31/713; C12Q 1/68; A61K 39/00

(52) U.S. Cl. CPC G01N 33/57492; G01N 2500/04; G01N 33/57419; C07K 14/705; C07K 16/28; A61K 31/713; C12Q 1/6886; G01N 33/57446; G01N 33/57407; G01N 33/57438; A61K 2039/505; C07K 2317/73; C12Q 2600/136; C12Q 2600/158; G01N 2333/705; G01N 2500/00; G01N 2800/50; A61K 39/39558

(57) ABSTRACT

The present invention provides a novel cancer marker for testing a morbidity risk of a cancer. The cancer marker according to the present invention is a prorenin receptor. A test method for testing a morbidity risk of a cancer according to the present invention includes measuring a prorenin receptor expression in a biological specimen obtained from a subject and further includes, for example, a step of comparing a prorenin receptor expression level in the biological specimen obtained from the subject with a reference value to test the morbidity risk of the cancer in the subject. The reference value is a prorenin receptor expression level in a biological specimen obtained from a healthy subject or a cancer patient. When the expression level in the subject is higher than that in the healthy subject or identical to or higher than that in the cancer patient, it can be evaluated that the subject has the mobility risk of the cancer.

### TECHNICAL FIELD

The present invention relates to a cancer marker, a test method for testing a morbidity risk of a cancer using the same, and a test reagent, and further relates to a cancer therapeutic drug and a screening method for the same.

### BACKGROUND ART

The commonest cause of death of Japanese has been cancer recently, and the leading cause of death in other countries also has been cancer. The emergence and the progression of cancer are related to various factors such as a genetic factor and an environmental factor. However, the definitive diagnosis and treatment methods have not been established yet.

Among cancers, for example, a pancreatic cancer rapidly progresses, and thus, it is difficult to detect the cancer early, and at the time when the cancer is detected, it has already metastasized to the surrounding lymph node, organ, and the like in many cases. As described above, the cancer has already progressed at the stage of therapy. Thus, for example, there is a problem in that it is difficult to treat the cancer, and even when a surgery is performed, the prognosis is bad. Therefore, it is desired to develop a cancer marker that allows various cancers including the pancreatic cancer to be detected early (Non-patent documents 1 and 2).

### PRIOR ART DOCUMENTS

### Non-Patent Document

Non-Patent Document 1: Duffy M J et al., “Tumor markers in pancreatic cancer: a European Group on Tumor Markers (EGTM) status report”, Annals of Oncology, Oxford Journal, March 2010, Vol. 21, No. 3, p. 441-447
Non-Patent Document 2: WATANABE Hiroyuki et at, “Feature, Diagnosis and treatment of pancreatic cancer, Is it possible to early diagnose pancreatic cancer? Tumor marker”, Geka chiryo (surgical therapy), Nagai Shoten Co., Ltd., September 2007, Vol. 97, p. 250-257

### SUMMARY OF INVENTION

### Problem to be Solved by the Invention

Hence, the present invention is intended to provide a novel cancer marker for testing a morbidity risk of a cancer.

### Means for Solving Problem

The cancer marker according to the present invention is a prorenin receptor.

The test method for testing a morbidity risk of a cancer according to the present invention includes the step of measuring a prorenin receptor expression level in a biological specimen obtained from a subject.

The test reagent according to the present invention is a test reagent for use in the test method for testing a morbidity risk of a cancer according to the present invention, including an expression measurement reagent for measuring a prorenin receptor expression.

The cancer therapeutic drug according to the present invention includes at least one of a binding substance for binding to a prorenin receptor and an expression suppressive substance for suppressing a prorenin receptor expression. The cancer therapeutic method for treating a cancer according to the present invention includes the step of administrating the cancer therapeutic drug according to the present invention to a subject.

The screening method according to the present invention is a screening method for a candidate substance for use in a cancer therapy, including selecting, from at least one target substance, a binding substance for binding to a prorenin receptor or an expression suppressive substance for suppressing a prorenin receptor expression as the candidate substance.

### Effects of the Invention

As the results of the earnest studies, the inventors of the present invention found that a prorenin receptor expression in a biological body showed a correlation with the occurrence of a cancer and thus arrived at the present invention. The present invention allows a morbidity risk of a cancer in a subject to be tested by measuring a prorenin receptor expression level. Moreover, in the present invention, the prorenin receptor becomes a target of a cancer. Thus, by screening using the target, a candidate substance for use in a cancer therapy can also be obtained. Therefore, the present invention is really useful in a clinical field and a biochemical field.

### BRIEF DESCRIPTION OF DRAWINGS

FIGS. 1A and 1B are graphs each showing the concentration of the prorenin receptor protein in the plasma in Example 1.

FIG. 2 is a graph showing the time-dependent change of the number of cells in Example 2.

FIG. 3 is a graph showing the expression of the prorenin receptor protein in Example 3.

FIG. 4 is a graph showing the expression of mRNA of the prorenin receptor gene in Example 3.

FIG. 5 is a graph showing the expression of the prorenin receptor protein in Example 4.

FIG. 6 is a graph showing the expression of mRNA of the prorenin receptor gene in Example 4.

FIG. 7 is a graph showing the expression of the prorenin receptor protein in Example 5.

FIG. 8 is a graph showing the expression of mRNA of the prorenin receptor gene in Example 5.

FIGS. 9A to 9D are tissue staining views each showing the expression of the prorenin receptor protein in Example 6.

FIGS. 10A and 10B are graphs each showing the tumor volume in Example 7.

FIGS. 11A and 11B are photographs of the mice in Example 7.

FIG. 12 is a graph showing the expression of the prorenin receptor protein in Example 7.

FIGS. 13A and 13B are graphs each showing the tumor volume in Example 8.

FIGS. 14A and 14B are photographs each showing the expression of the prorenin receptor protein in Example 9.

FIG. 15 is a photograph showing the expression of the prorenin receptor protein in Example 10.

FIG. 16 is a graph showing the expression of the phosphorylated LRP protein in Example 10.

FIGS. 17A to 17C are graphs each showing the expression of the active β-catenin protein and the Cyllin D1 protein in Example 10.

FIGS. 18A to 18C are graphs each showing the proliferation potency of cells in Example 11.

FIG. 19 shows dot plots each showing the proportion of the apoptosis cells in Example 12.

FIG. 20 is a histogram showing the proportion of the apoptosis cells in Example 12.

FIGS. 21A and 21B are graphs each showing the proportion of the cells in each cell cycle in Example 12.

FIGS. 22A to 22C are graphs each showing the activity of the caspase 3 in Example 12.

FIG. 23 is a graph showing the concentration of the prorenin receptor protein in plasma in Example 13.

### DESCRIPTION OF EMBODIMENTS

(Cancer Marker)

The cancer marker according to the present invention is, as mentioned above, a prorenin receptor. The cancer marker according to the present invention allows a morbidity risk of a cancer in a subject to be tested by measuring a prorenin receptor expression level in a biological specimen obtained from the subject, for example.

The origin of the prorenin receptor is not limited to particular origins and can be set appropriately according to the type of a subject. Examples of the origin include humans and nonhuman animals except humans. Examples of the nonhuman animals include mammals such as a mouse, a rat, a dog, a monkey, a rabbit, a sheep, and a horse. For prorenin receptors derived from various animals, information registered in an existing database can be referred to. Specific examples of the prorenin receptors derived from humans include, as cDNA, a region (including a stop codon) extending from 103rd to 1155th bases in the following base sequence (SEQ ID NO: 5) registered as the NCBI accession No. NM_005765, and as a protein, the following amino acid sequence (SEQ ID NO: 6) registered as the NCBI accession No. NP_005756. The base sequence of SEQ ID NO: 5 is a sequence which encodes the amino acid sequence of SEQ ID NO: 6.

Human prorenin receptor cDNA
(SEQ ID NO: 5)
ctggacgagtccgagcgcgtcacctcctcacgctgcggctgtcgcccgtg
tcccgccggcccgttccgtgtcgccccgcagtgctgcggccgccgcggca
ccatggctgtgtttgtcgtgctcctggcgttggtggcgggtgattgggga
acgagtttagtatattaaaatcaccagggtctgttgattccgaaatggaa
attggcctataccaggagagcggatcccagacgtggctgcattgtccatg
ggcttctctgtgaaagaagacctttcttggccaggactcgcagtgggtaa
cctgtttcatcgtcctcgggctaccgtcatggtgatggtgaagggagtga
acaaactggctctacccccaggcagtgtcatttcgtaccctttggagaat
gcagttccttttagtcttgacagtgttgcaaattccattcactccttatt
ttctgaggaaactcctgttgattgcagttggctcccagtgaggaaagagt
gtatatggtagggaaggcaaactcagtgtttgaagacctttcagtcacct
tgcgccagctccgtaatcgcctgtttcaagaaaactctgttctcagttca
ctccccctcaattctctgagtaggaacaatgaagttgacctgctctttct
ttctgaactgcaagtgctacatgatatttcaagcttgctgtctcgtcata
agcatctagccaaggatcattctcctgatttatattcactggagctggca
ggtttggatgaaattgggaagcgttatggggaagactctgaacaattcag
agatgcttctaagatccttgttgacgctctgcaaaagtttgcagatgaca
tgtacagtctttatggtgggaatgcagtggtagagttagtcactgtcaag
tcatttgacacctccctcattaggaagacaaggactatccttgaggcaaa
acaagcgaagaacccagcaagtccctataaccttgcatataagtataatt
ttgaatattccgtggttttcaacatggtactttggataatgatcgccttg
gccttggctgtgattatcacctcttacaatatttggaacatggatcctgg
atatgatagcatcatttataggatgacaaaccagaagattcgaatggatt
gaatgttacctgtgccagaattagaaaagggggttggaaattggctgatt
gttaaaatatatcttttagtgtgctttaaagtagatagtatactttacat
ttataaaaaaaaatcaaattttgttattattttgtgtgtgcctgtgatga
tttctagagtgaattatagtattgacgtgaatcccactgtggtatagatt
ccataatatgatgaatattatgatatagccatttaataacattgatttca
ttctgtttaatgaatttggaaatatgcactgaaagaaatgtaaaacattt
agaatagctcgtgttatggaaaaaagtgcactgaatttattagacaaact
tacgaatgataacttattacacagcataggtgaaaatcatatttgggcta
ttgtatactatgaacaatttgtaaatgtcttaatttgatgtaaataactc
tgaaacaagagaaaaggtttttaacttagagtagccctaaaatatggatg
tgcttatataatcgcttagttttggaactgtatctgagtaacagaggaca
gctgattttaaccctcttctgcaagtttgttgacctacatgggctaatat
ggatactaaaaatactacattgatctaagaagaaactagccttgtggagt
atatagatgcttttcattatacacacaaaaatccctgagggacattttga
ggcatgaatataaaacatttttatttcagtaacttttccccctgtgtaag
ttactatggtttgtggtacaacttcattctatagaatattaagtggaagt
gggtgaattctactttttatgttggagtggaccaatgtctatcaagagtg
acaaataaagttaatgatgattccaaaaaaaaaa
Human prorenin receptor protein
(SEQ ID NO: 6)
MAVFVVLLALVAGVLGNEFSILKSPGSVVFRNGNWPIPGERIPDVAALSM
GFSVKEDLSWPGLAVGNLFHRPRATVMVMVKGVNKLALPPGSVISYPLEN
AVPFSLDSVANSIHSLFSEETPVVLQLAPSEERVYMVGKANSVFEDLSVT
LRQLRKRLFQENSVLSSLPLNSHSRNNEVDLLFLSELQVLHDISSLLSRH
KHLAKDHSPDLYSLELAGLDEIGKRYGEDSEQFRDASKILVDALQKFADD
MYSLYGGNAVVELVTVKSFDTSLIRKTRTILEAKQAKNPASPYNLAYKYN
FEYSVVFNMVLWIMIALALAVIITSYNIWNMDPGYDSITYRMTNQKIRMD

The prorenin receptor can be used as a cancer marker and can be used specifically as a cancer marker for a gastrointestinal cancer and a brain tumor, for example. Examples of the gastrointestinal cancer include a gastrointestinal tract cancer and a digestive gland cancer. Examples of the gastrointestinal tract cancer include a gastric cancer and a large intestine cancer, and examples of the digestive gland cancer include a pancreatic cancer and a liver cancer. Examples of the brain tumor include glioma, astrocytoma, primary central nervous system malignant lymphoma, and cavernous hemangioma. Besides these, the cancer marker according to the present invention can be a cancer marker for a peritoneal cancer, for example. Moreover, the present invention allows both of a primary cancer and a metastatic cancer to be tested, for example.

The marker according to the present invention may be, for example, a prorenin receptor protein or mRNA transcribed from a prorenin receptor gene.

(Test Method for Testing Morbidity Risk of Cancer)

The test method for testing a morbidity risk of a cancer according to the present invention includes the step of measuring a prorenin receptor expression level in a biological specimen obtained from a subject, as mentioned above.

The present invention is characterized in that a prorenin receptor expression level is measured as a cancer marker, and the other steps and conditions are not limited to particular steps and conditions.

The test method according to the present invention allows the possibility of the occurrence of a cancer, the presence or absence of the occurrence of a cancer (whether or not malignant transformation occurs), the stage of a cancer, the state of prognosis, and the like to be evaluated, for example. Examples of a cancer to be tested include a gastrointestinal cancer and a brain tumor as mentioned above. Examples of the gastrointestinal cancer include a gastrointestinal tract cancer and a digestive gland cancer, examples of the gastrointestinal tract cancer include a gastric cancer and a large intestine cancer, and examples of the digestive gland cancer include a pancreatic cancer and a liver cancer. Examples of the brain tumor include glioma, astrocytoma, primary central nervous system malignant lymphoma, and cavernous hemangioma. The test method according to the present invention also allows a peritoneal cancer to be evaluated, for example. The present invention also allows both of a primary cancer and a metastatic cancer to be tested, for example.

In the test method according to the present invention, examples of the subject include humans and nonhuman animals except humans. Examples of the nonhuman animals include mammals such as a mouse, a rat, a dog, a monkey, a rabbit, a sheep, and a horse.

In the test method according to the present invention, the type of the biological specimen is not limited to particular specimens, and examples thereof include a body fluid, a cell derived from a body fluid, an organ, a tissue, and a cell, separated from a biological body. The body fluid can be, for example, blood, and specific examples thereof include whole blood, serum, and plasma. The cell derived from a body fluid can be, for example, a cell derived from blood, and specific examples thereof include blood cells such as a hemocyte, a leukocyte, and a lymphocyte. The biological specimen can be decided appropriately according to the type of a cancer to be tested, for example. The biological specimen is derived from an organ in which a cancer to be tested emerges, for example. Examples of the organ include a stomach, a pancreas, a large intestine, and a liver, a brain, and a peritoneum. Examples of the brain include a cerebrum, a temporal lobe, an occipital lobe, a cerebellum, a basal ganglion, and a mesenchymal tissue. As a specific example, in the case where the cancer to be tested is a pancreatic cancer, the biological specimen is preferably a tissue or a cell derived from a pancreas. In the case where the cancer to be tested is a brain tumor, the biological specimen is preferably a tissue or a cell derived from a brain. The cancer marker according to the present invention allows a cancer in a digestive organ such as a pancreas and a brain such as a cerebrum to be tested by a prorenin receptor expression level in blood, for example. Thus, the biological specimen is preferably whole blood, serum, or plasma, more preferably serum or plasma because burdens on subjects and doctors can be eased, for example.

A prorenin receptor expression to be measured can be, for example, an expression of mRNA of a prorenin receptor gene or a prorenin receptor protein. Either one of the expressions of the mRNA and the protein in the biological specimen may be measured, or both of them may be measured, for example. The methods for measuring these are not limited to particular methods, and known methods can be employed. Specific examples of the method for measuring the expression of the mRNA include gene amplification methods utilizing a reverse transcription reaction such as a reverse transcription (RT)-PCR method and the like. Specifically, for example, the method is a method in which cDNA is synthesized from mRNA by a reverse transcription reaction, and a gene amplification is performed using the cDNA as a template. Examples of the method for measuring the expression of the protein include an immune antibody method, an ELISA method, flow cytometry, and Western blotting.

The test method according to the present invention further includes the step of comparing a prorenin receptor expression level in the biological specimen (hereinafter also referred to the “biological specimen to be tested”) obtained from the subject with a reference value to test the morbidity risk of the cancer in the subject. The reference value is not limited to particular values, and examples thereof include the prorenin receptor expression levels in a healthy subject, a cancer patient, and a cancer patient in each stage of progress. In the case of evaluating the prognosis, the reference value may be the prorenin receptor expression level after therapy (for example, immediately after therapy) in the same subject, for example.

The reference value can be obtained using a biological specimen (hereinafter also referred to as a “reference biological specimen”) isolated from a healthy subject and/or a cancer patient as mentioned above, for example. In the case of evaluating the prognosis, a reference biological specimen isolated from the same subject after therapy may be used, for example. The reference value may be measured at the same time as the measurement of the biological specimen to be tested obtained from the subject or in advance. The latter case is preferable because it is not necessary to obtain the reference value every time the measurement of the biological specimen to be tested obtained from the subject. It is preferred that the biological specimen to be tested obtained from the subject and the reference biological specimen are collected under the same conditions and subjected to the measurement of a prorenin receptor under the same conditions, for example.

In the step of comparing, a method for evaluating a morbidity risk of a cancer in a subject is not limited to particular methods and can be decided appropriately according to the type of the reference value. As a specific example, when the prorenin receptor expression level in a biological specimen to be tested obtained from a subject is significantly higher than that in a reference biological specimen obtained from a healthy subject, identical to (has no significant difference from) that in a reference biological specimen obtained from a cancer patient, and/or is significantly higher than that in a reference biological specimen obtained from a cancer patient, it can be evaluated that the subject has a morbidity risk or a high morbidity risk of a cancer. When the prorenin receptor expression level in a biological specimen to be tested obtained from a subject is identical to (has no significant difference from) that in a reference biological specimen obtained from a healthy subject, is significantly lower than that in a reference biological specimen obtained from a healthy subject, and/or is significantly lower than that in a reference biological specimen obtained from a cancer patient, it can be evaluated that the subject has no morbidity risk or a low morbidity risk of a cancer. In the step of comparing, comparing the prorenin receptor expression level in a biological specimen to be tested obtained from a subject with that in a reference biological specimen obtained from a cancer patient in each stage of progress allows the degree of progression of a cancer to be evaluated. Specifically, when the expression level in a biological specimen to be tested obtained from a subject is, for example, almost identical to (has no significant difference from) that in the reference biological specimen in any stage of progression, it can be evaluated that the subject has a possibility of being in the stage of progression.

In the case where the state of prognosis is evaluated in the step of comparing, the evaluation may be performed in the same manner as mentioned above or can be performed using, as a reference value, the prorenin receptor expression level in a reference biological specimen obtained from the same subject after therapy, for example. As a specific example, when the prorenin receptor expression level in a biological specimen to be tested obtained from a subject is significantly higher than the reference value, it can be evaluated that the subject has a risk of relapse or deterioration after therapy. When the prorenin receptor expression level in a biological specimen to be tested obtained from a subject is identical to (has no significant difference from) the reference value and/or is significantly lower than the reference value, it can be evaluated that the subject has no risk or a low risk of relapse after therapy.

In the present invention, the prorenin receptor expression levels in biological specimens collected over time from the same subject may be compared, for example. Then, for example, when the expression level is increased over time, it can be determined that the morbidity risk is increased and the like, and when the expression level is decreased over time, it can be determined that the morbidity risk is decreased, the cancer is cured, and the like.

(Test Reagent)

The test reagent according to the present invention is a test reagent for use in the test method for testing a morbidity risk of a cancer according to the present invention, including an expression measurement reagent for measuring a prorenin receptor expression. The test reagent according to the present invention allows the test method for testing a morbidity risk of a cancer according to the present invention to be performed easily. The present invention is characterized in that a test of a morbidity risk of a cancer is performed on the basis of the measurement of a prorenin receptor expression and is only required that the prorenin receptor expression is measured, and the type of the expression measurement reagent is not limited to particular reagents. The expression measurement reagent for measuring a prorenin receptor expression may be, for example, a reagent for measuring an expression of a prorenin receptor protein or mRNA of a prorenin receptor gene.

The former can be, for example, a substance for binding to a prorenin receptor protein, and a specific example thereof can be an antibody. In this case, the test reagent according to the present invention preferably further includes a detection reagent for detecting a binding between the prorenin receptor protein and the antibody, for example. The detection reagent can be, for example, a combination of a detectable labeled antibody to the antibody and a substrate to the label.

The latter can be, for example, a reagent for amplifying mRNA of a prorenin receptor gene by a reverse transcription, and a specific example thereof can be, for example, a primer. The primer can be designed appropriately based on a gene sequence of a prorenin receptor, for example.

(Cancer Diagnostic Method and Cancer Diagnostic Reagent)

A cancer diagnostic method for diagnosing a cancer according to the present invention includes the step of measuring a prorenin receptor expression level in a biological specimen obtained from a subject. A cancer diagnostic reagent for diagnosing a cancer according to the present invention includes an expression measurement reagent for measuring a prorenin receptor expression. The present invention can be described with reference to the descriptions of the test method and the test reagent according to the present invention.

(Cancer Therapeutic Drug and Cancer Therapeutic Method)

A target of a cancer according to the present invention is a prorenin receptor. In the present invention, a prorenin receptor is used as the target of a cancer, and for example, the cancer can be treated by suppressing an expression of mRNA of a prorenin receptor gene or a prorenin receptor protein or suppressing or neutralizing a function of a prorenin receptor protein, and the like.

The cancer therapeutic drug according to the present invention includes at least one of a binding substance for binding to a prorenin receptor and an expression suppressive substance for suppressing a prorenin receptor expression as mentioned above. The cancer therapeutic method for treating a cancer according to the present invention includes the step of administering the cancer therapeutic drug according to the present invention.

The present invention is characterized in that a cancer is treated by binding of a binding substance to a prorenin receptor protein or suppression of a prorenin receptor expression, and the types of the binding substance and the expression suppressive substance are not limited to particular substances. In the present invention, for example, either one or both of the binding substance and the expression suppressive substance may be used.

Examples of the binding substance include a low molecular weight compound, a peptide, a protein, and a nucleic acid, and the binding substance is preferably an antibody or an antigen binding fragment thereof. The binding substance is preferably an antagonist.

The antibody is not limited to particular antibodies and can be obtained by a general method for producing an antibody. As a specific example, an animal is immunized with a prorenin receptor protein or a fragment thereof as an antigen, and serum is collected from the immunized animal, and thus, an antibody to the prorenin receptor protein can be obtained. The antibody may be, for example, a monoclonal antibody or a polyclonal antibody.

The antigen may be, for example, a full-length amino acid sequence of a prorenin receptor or a partial sequence thereof as mentioned above. The partial sequence can be, for example, a region extending from the 200th to 213rd amino acid residues (HKHLAKDHSPDLYS: SEQ ID NO: 7) in an amino acid sequence (SEQ ID NO: 6) of a human prorenin receptor protein, for example.

In the prorenin receptor protein, a position of binding between the prorenin receptor protein and an antibody to the prorenin receptor protein is not limited to particular positions. In the case where the prorenin receptor protein is a human prorenin receptor protein, the position of the binding can be, for example, a region extending from 200th to 213rd amino acid residues (HKHLAKDHSPDLYS: SEQ ID NO: 7) in the amino acid sequence (SEQ ID NO: 6) of the human prorenin receptor protein.

Examples of the expression suppressive substance include a substance for suppressing a transcription of mRNA from a prorenin receptor gene, a substance for cleaving transcribed mRNA, and a substance for suppressing a translation of a protein from mRNA. Specific examples thereof include an RNA interference reagent such as siRNA, an antisense, and a ribozyme, and they may be used alone or in a combination of two or more of them.

The conditions under which the cancer therapeutic drug according to the present invention is administrated are not limited to particular conditions, and the type of administration, the timing of administration, the amount of administration, and the like can be set appropriately according to the type of a target cancer disease, the degree of progress, the age of the subject, and the like. Examples of the target of the administration include humans and nonhuman animals except humans. Examples of the nonhuman animals include mammals such as a mouse, a rat, a dog, a monkey, a rabbit, a sheep, and a horse.

(Screening Method)

The screening method according to the present invention is a screening method for a cancer therapeutic drug candidate substance(s), including selecting, from a target substance(s), a binding substance for binding to a prorenin receptor or an expression suppressive substance for suppressing a prorenin receptor expression as the cancer therapeutic drug candidate substance(s). The present invention is characterized in that a target of the cancer therapeutic drug candidate substance is a prorenin receptor, and the other steps and conditions are not limited to particular steps and conditions.

Examples of the binding substance include a low molecular weight compound, a peptide, a protein, and a nucleic acid, and the binding substance is preferably an antibody or an antigen binding fragment.

Examples of the expression suppressive substance include a substance for suppressing a transcription of mRNA from a prorenin receptor gene, a substance for cleaving transcribed mRNA, and a substance for suppressing a translation of a protein from mRNA. Specific examples thereof include an RNA interference reagent such as siRNA, an antisense, and a ribozyme.

A screening method for the binding substance according to the present invention includes the steps of causing a target substance(s) to be in contact with a prorenin receptor; detecting a binding between the prorenin receptor and the target substance(s); and selecting the target substance(s) binding to a prorenin receptor as the cancer therapeutic drug candidate substance(s). In the step of detecting, a method for detecting a binding between the prorenin receptor and the target substance(s) is not limited to particular methods and can be decided appropriately according to the type of the target substance(s), for example.

A screening method for the expression suppressive substance according to the present invention includes the steps of: causing a target substance(s) to be present in an expression system of the prorenin receptor to cause a prorenin receptor expression; detecting the prorenin receptor expression in the expression system; and selecting the target substance(s) having a prorenin receptor expression level lower than an expression system of a control including no target substance present therein as the candidate substance(s), for example. In the step of detecting, the expression to be detected may be, for example, an expression of a prorenin receptor protein or a transcription of mRNA of a prorenin receptor gene. Methods for detecting the expression of the protein and the expression of the mRNA is not limited to particular methods, and known methods can be employed.

### EXAMPLES

The examples of the present invention will be described below. It is to be noted, however, that the present invention is not limited by the following examples.

### Example 1

The increase in the concentration of the prorenin receptor in the plasma of pancreatic cancer patients was examined.

The measurement of the prorenin receptor in the plasma was performed using Human soluble (Pro)renin Receptor Assay Kit (product of Immuno-Biological Laboratories Co. Ltd) according to its protocol. First, plasma was collected from the blood of each of the following subjects: healthy males (n=9), healthy females (n=3), male pancreatic cancer patients (n=9), and female pancreatic cancer patients (n=8). The collected plasma was diluted 2-fold with the diluent (phosphate buffer solution that contains 1% BSA and 5% Tween-20) of the kit, and the diluted plasma thus obtained was used as a test sample. 100 μl of the test sample was added to a plate to which a capture antibody (anti-Human renin receptor polyclonal rabbit IgG antibody) is bound, and allowed to react at 4° C. overnight. The test sample was removed and the plate was washed four times with the phosphate buffer solution. Next, 100 μl of labeled antibody (HRP-labeled anti-Human renin receptor polyclonal mouse IgG antibody) was added to the plate and allowed to react at 4° C. for 60 minutes. The labeled antibody was removed and the plate was washed five times with the phosphate buffer solution. Furthermore, after developing the color by adding 100 μl of a Tetra Methyl Benzidine-containing substrate solution to the plate, the color development reaction was stopped with 0.5 mold (1N) H₂SO₄. After stopping the reaction, the absorbance at 450 nm regarding the reacted solution in the plate was measured by a plate reader. On the other hand, the standard (Human soluble (Pro)renin Receptor) of the kit was serially diluted, and the absorbance was measured in the same manner using each of the thus obtained diluted standard samples to make a calibration curve. Then, from the absorbance of the test sample, the concentration of the prorenin receptor in the plasma was measured on the basis of the calibration curve.

Also plasma was collected from the following subjects: healthy subjects (n=20), pancreatic cancer patients of primary pancreatic cancer without metastasis (no metastasis, n=11), and pancreatic cancer patients of primary pancreatic cancer with metastases to other organs (with metastases, n=9), and the concentration of the prorenin receptor in the plasma was measured in the same manner.

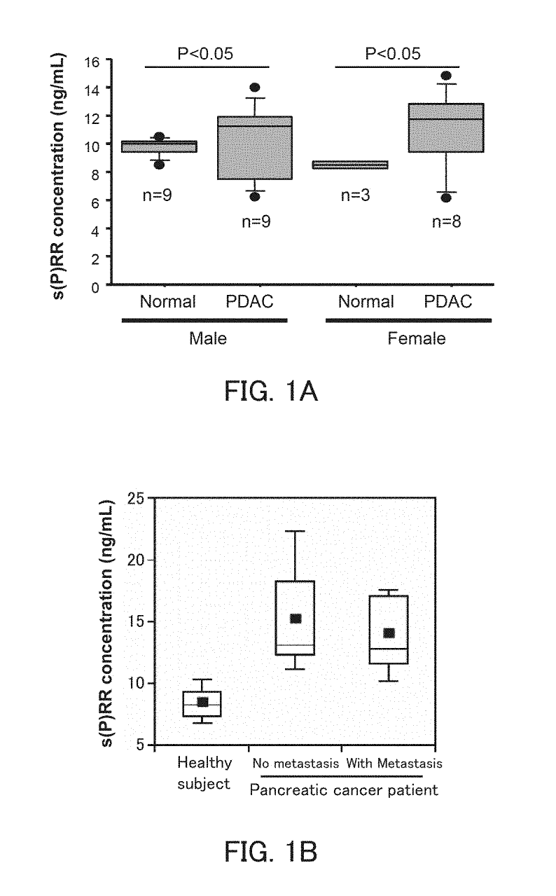

The results thereof are shown in FIGS. 1A and 1B. FIGS. 1A and 1B are graphs each showing the concentration of the prorenin receptor in the plasma. In FIGS. 1A and 1B, each of the horizontal axes indicates the types of the test samples and each of the vertical axes indicates the concentration of the prorenin receptor. As can be seen from FIG. 1A, in both male and female subjects, the concentrations of the prorenin receptor in the plasma of the pancreatic cancer patients (PDAC) were significantly higher than those in the plasma of the healthy subjects (Normal). As can be seen from FIG. 1B, in both the pancreatic cancer patients without metastasis and the pancreatic cancer patients with metastases, the concentrations of the prorenin receptor in the plasma were significantly higher than those in the healthy subjects. From these results, it was found that there is a correlation between the occurrence of the pancreatic cancer and the increase in the concentration of the prorenin receptor in the plasma regardless of the presence or absence of metastasis or sex, which demonstrates that the prorenin receptor in the plasma serves as a pancreatic cancer marker.

### Example 2

The prorenin receptor expression in pancreatic cancer cell lines was suppressed and the proliferative potency of the cell line was examined.

Human pancreatic cancer cell line PK-1 with 40% confluence was cultured in a serum-free culture medium on a dish under the conditions of 37° C. and 5% CO₂ for 24 hours. Next, PK-1 was transfected with the following siRNA (P) RR (product of Life Technologies) for causing knockdown of a prorenin receptor gene or scrambled siRNA (product of Life Technologies), which is a negative control, using Lipofectamine (trademark) RNAiMAX (product of Life Technologies) according to its protocol. Then, siRNA-transfected PK-1 was cultured under the same conditions for 48 hours. After the culture, PK-1 was peeled from the dish using 0.25% trypsin and 1 mmol/1 EDTA-containing phosphate buffer solution (product of Life Technologies), and the serum-free culture medium was newly added. Then, from the culture medium that contains PK-1, a pellet that contains PK-1 was collected by centrifugation. The pellet was suspended in 1 ml of phosphate buffer solution, the suspension thus obtained and 20 μl of trypan blue solution were mixed, and the resultant was injected into a hemocytometer to calculate the number of cells per 1 ml. The resultant was seeded so as to achieve the number of living cells of 5×10⁵ cells/10 cm-diameter dish, and human Wnt3a (product of R&D Systems) for inducing cell proliferation was added at 150 ng/ml. After culturing for 0, 24, 48, and 72 hours, PK-1 was collected and the number of living cells was calculated in the same manner. Also, as a control, the culture and the measurement of the number of living cells were performed in the same manner except that the cell line was not treated with siRNA.

siRNA (P) RR
Sense strand
(SEQ ID NO: 8)
5′-UAUAGGGACUUGCUGGGUUCUUCGC-3′
Antisense strand
(SEQ ID NO: 9)
5′-GCGAAGAACCCAGCAAGUCCCUAUA-3′

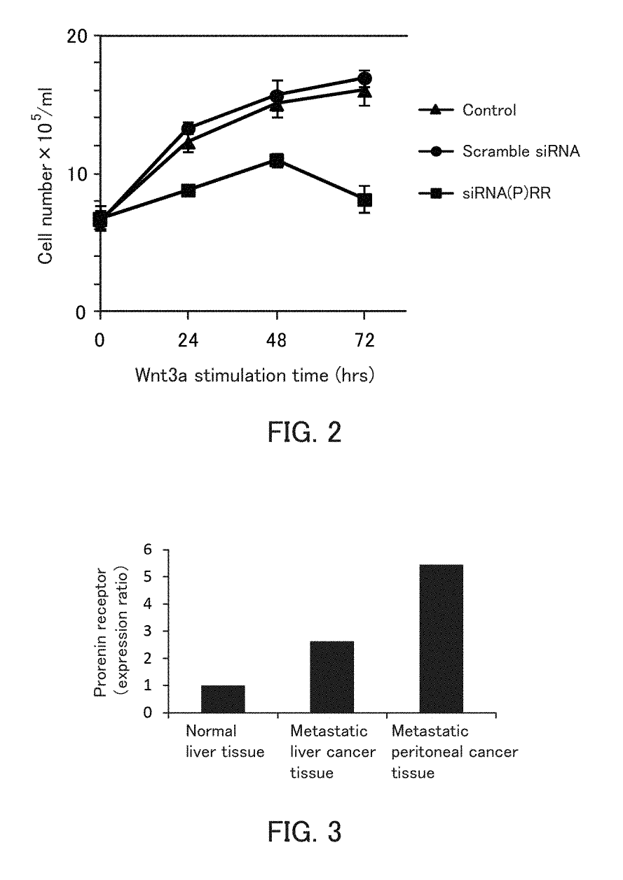

The results thereof are shown in FIG. 2. FIG. 2 is a graph showing the number of cells of the pancreatic cancer cell line when the prorenin receptor expression is suppressed by RNAi. In FIG. 2, the control (filled circle) indicates the results of a control group not treated with siRNA, the scramble (filled triangle) indicates the results of a scramble group treated with scramble siRNA, and siRNA (P) RR (filled square) indicates the results of a siRNA (P) RR group treated with siRNA (P) RR. In FIG. 2, the horizontal axis indicates the time stimulated by Wnt3a and the vertical axis indicates the number of cells. As can be seen from FIG. 2, the control group and the scramble group showed comparable cell proliferation at each processing time. In contrast, the cell proliferation of the pancreatic cancer cell line of the siRNA (P) RR group was suppressed as compared with that of the control group and the scramble group at every processing time. Furthermore, the cells in the control group and the scramble group were continuously proliferated up to 72 hours whereas the number of cells in the siRNA (P) RR group was decreased after peaking at 48 hours. From this result, it was found that the proliferation of the pancreatic cancer cell line can be suppressed by suppressing the prorenin receptor expression. That is, this result showed that the prorenin receptor can be a pancreatic cancer target.

### Example 3

The increase in the expression of the prorenin receptor protein and mRNA in metastatic cancer tissues was examined.

(1) Expression of Prorenin Receptor Protein

From cancer patients with primary pancreas cancer, normal liver tissues (control), metastatic liver cancer tissues, and metastatic peritoneal cancer tissues were collected. 1 ml of Lysis buffer was added to 2 g of each tissue, and the resultant was subjected to homogenization at 4,000 rpm for 5 minutes to prepare a test sample. The amount of the protein in the test sample was measured using a reagent (trade name: Bio-Rad Protein Assay (product of Bio-Rad)).

The test sample (total protein amount: 20 μg) was subjected to electrophoresis using 10% SDS polyacrylamide gel and transcribed to a polyvinylidene difluoride (PVDF) membrane. The membrane after the transcription was subjected to blocking using Blocking buffer (product of Li-Cor BioSciences). The membrane after the blocking was allowed to react with 1000-fold diluted anti-rabbit (P) RR polyclonal antibody at room temperature for 1 hour, followed by reaction with 2000-fold diluted IRDye (registered trademark)-labeled anti-rabbit IgG antibody (product of Li-Cor BioSciences) under the same conditions. Regarding the membrane after reaction, the expression level of the prorenin receptor protein in the test sample was measured using Odyssey (registered trademark) System (product of Li-Cor BioSciences). The relative value of the expression levels of the metastatic liver cancer tissue and the metastatic peritoneal cancer tissue was obtained assuming that the expression level of the normal liver tissue, which is a control, was 1.

The results of the expression of the prorenin receptor protein in the respective tissues are shown in FIG. 3. FIG. 3 is a graph showing the expression levels of the prorenin receptor proteins in the respective tissues. The horizontal axis indicates the types of test samples and the vertical axis indicates the relative expression level of the prorenin receptor protein. As can be seen from FIG. 3, the expression of the prorenin receptor protein in the metastatic liver cancer tissues and the metastatic peritoneal cancer tissues was significantly higher than that in the normal liver tissues. From these results, it was found that there is a correlation between the metastatic cancer tissue and the increase in the expression of the prorenin receptor protein, which demonstrates that the prorenin receptor protein serves as a metastatic cancer marker.

(2) Expression of mRNA of Prorenin Receptor Gene

The normal liver tissues (control), the metastatic liver cancer tissues, and the metastatic peritoneal cancer tissues collected in the above item (1) were used. 1 ml of ISOGEN (product of Nippon Gene) was added to 2 g of each tissue, and subjected to homogenization at 4,000 rpm for 5 minutes to prepare a test sample. Ethanol was added to the test sample so as to precipitate RNA, and the RNA was dissolved in RNase free water to prepare a RNA solution. The concentration and OD₂₆₀/OD₂₈₀ (purity) of the RNA solution were measured by a spectrophotometer. Then, the RNA solution having a RNA concentration of 300 ng/μl with a purity of 1.8 or more was used for the quantitative real time (qRT)-PCR below.

After synthesizing cDNA from the RNA solution by a routine procedure using reverse transcriptase and random primers, RNA was degraded using RNaseH. With the cDNA thus obtained being used as a template, qRT-PCR was performed using SYBR (registered trademark) Green (product of Applied Biosystems) and Applied Biosystems 7300 Real-Time PCR System (product of Applied Biosystems) according to the attached protocols, and the expression level of mRNA of the prorenin receptor gene in the test sample and the expression level of mRNA of the β-actin gene, which is an internal standard, were measured. The expression level of mRNA of the prorenin receptor gene was calculated as a ratio to the expression level of mRNA of the β-actin gene. In the qRT-PCR, the respective primer sets below were used for amplifying the prorenin receptor gene and the β-actin gene.

Prorenin receptor gene amplification primer set
(SEQ ID NO: 1)
5′-GGCGTTGGTGGCGGGTGTTT-3′
(SEQ ID NO: 2)
5′-AGCCCATGGACAATGCAGCCAC-3′
β-actin gene amplification primer set
(SEQ ID NO: 3)
5′-CACAGAGCCTCGCCTTTGCCGATC-3′
(SEQ ID NO: 4)
5′-ACGAGCGCGGCGATATCATCATC-3′

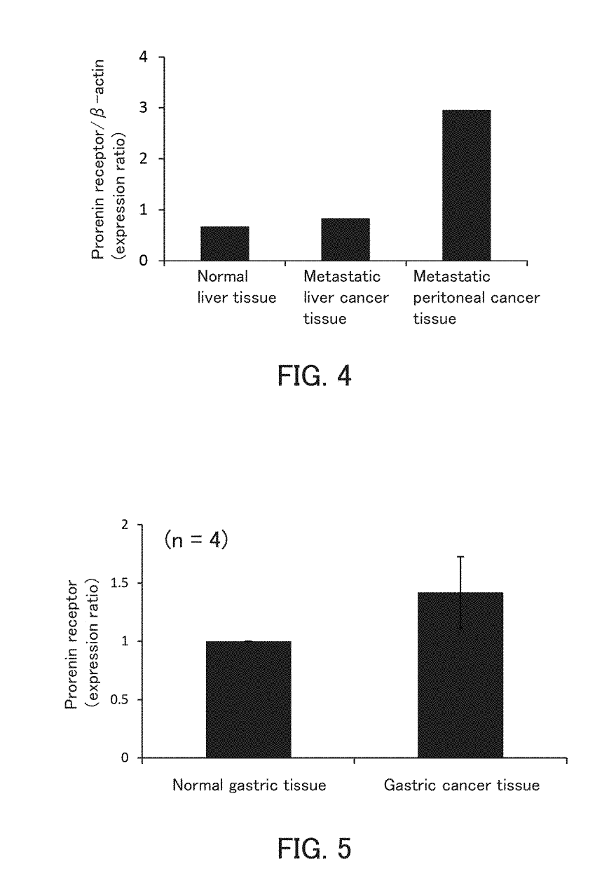

The results of the expression levels of mRNA of the prorenin receptor gene in the respective tissues are shown in FIG. 4. The horizontal axis indicates the types of test samples and the vertical axis indicates the relative expression level of mRNA of the prorenin receptor gene to mRNA of the β-actin gene. As can be seen from FIG. 4, the expression of mRNA of the prorenin receptor gene in the metastatic liver cancer tissues and the metastatic peritoneal cancer tissues was higher than that in the normal liver tissues. From this result, it was found that there is a correlation between the metastatic cancer tissue and the increase in the expression of mRNA of the prorenin receptor gene, which demonstrates that mRNA of the prorenin receptor gene serves as a metastatic cancer marker.

### Example 4

The increase in the expression of the prorenin receptor protein and mRNA in gastric cancer tissues was examined.

(1) Expression of Prorenin Receptor Protein

Normal gastric tissues (n=4) and gastric cancer tissues (n=4) were collected from four gastric cancer patients, and the expression level of the prorenin receptor protein was measured in the same manner as in the item (1) in Example 3. Then, the average for the expression levels was obtained regarding each of the normal tissues and the cancer tissues of four subjects.

The results of the expression of the prorenin receptor protein in the gastric cancer tissue are shown in FIG. 5. FIG. 5 is a graph showing the expression level of the prorenin receptor protein. The horizontal axis indicates the types of test samples and the vertical axis indicates the relative expression level of the prorenin receptor protein. As can be seen from FIG. 5, the expression of the prorenin receptor protein in the gastric cancer tissues (n=4) was significantly higher than that in the normal gastric tissues (n=4). From these results, it was found that there is a correlation between the occurrence of the gastric cancer and the increase in the expression of the prorenin receptor protein, which demonstrates that the prorenin receptor protein in the gastric tissue serves as a gastric cancer marker.

(2) Expression of mRNA of Prorenin Receptor Gene

The normal gastric tissues and the gastric cancer tissues collected in the above item (1) were used, and the expression level of mRNA of the prorenin receptor gene was measured in the same manner as in Example 3(2). Then, the average for the expression levels was obtained regarding each of the normal tissues and the cancer tissues of four subjects.

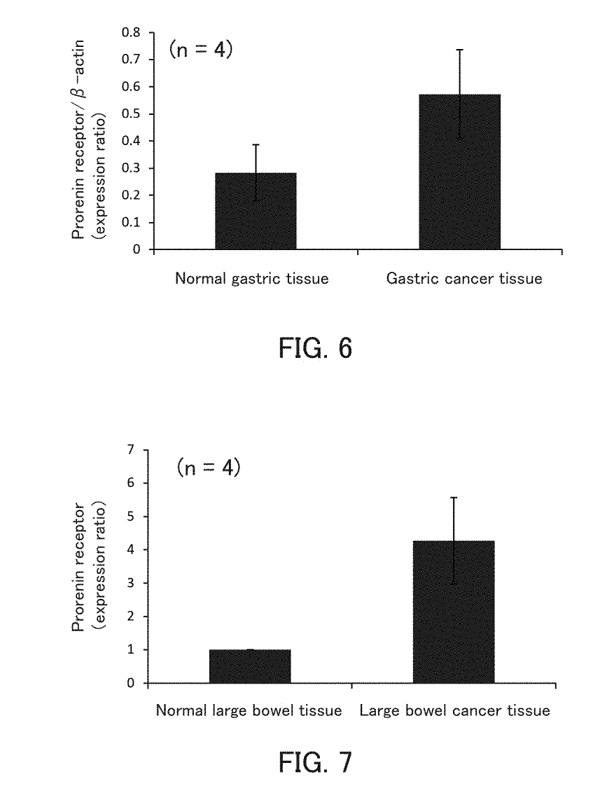

The results of the expression level of mRNA of the prorenin receptor gene in the gastric cancer tissue are shown in FIG. 6. The horizontal axis indicates the types of test samples and the vertical axis indicates the relative expression level of mRNA of the prorenin receptor gene to mRNA of the β-actin gene. As can be seen from FIG. 6, the expression of mRNA of the prorenin receptor gene in the gastric cancer tissues (n=4) was higher than that in the normal gastric tissues (n=4). From this result, it was found that there is a correlation between the occurrence of the gastric cancer and the increase in the expression of mRNA of the prorenin receptor gene, which demonstrates that the mRNA of the prorenin receptor gene in the gastric tissue serves as a gastric cancer marker.

### Example 5

The increase in the expression of the prorenin receptor protein and mRNA in large intestine cancer tissues was examined.

(1) Expression of Prorenin Receptor Protein

Normal large intestine tissues (n=4) and large intestine cancer tissues (n=4) were collected from four large intestine cancer patients, and the expression level of the prorenin receptor protein was measured in the same manner as in the item (1) in Example 3. Then, the average for the expression levels was obtained regarding each of the normal tissues and the cancer tissues of four subjects.

The results of the expression of the prorenin receptor protein in the large intestine cancer tissue are shown in FIG. 7. FIG. 7 is a graph showing the expression level of the prorenin receptor protein. The horizontal axis indicates the types of test samples and the vertical axis indicates the relative expression level of the prorenin receptor protein. As can be seen from FIG. 7, the expression of the prorenin receptor protein in the large intestine cancer tissues (n=4) was significantly higher than that in the normal large intestine tissues (n=4). From these results, it was found that there is a correlation between the occurrence of the large intestine cancer and the increase in the expression of the prorenin receptor protein, which demonstrates that the prorenin receptor protein in the large intestine tissue serves as a large intestine cancer marker.

(2) Expression of mRNA of Prorenin Receptor Gene

Regarding the normal large intestine tissues and the large intestine cancer tissues collected in the above item (1), the expression level of mRNA of the prorenin receptor gene was measured in the same manner as in Example 3(2). Then, the average for the expression levels was obtained respectively regarding the normal tissues and the cancer tissues of four subjects.

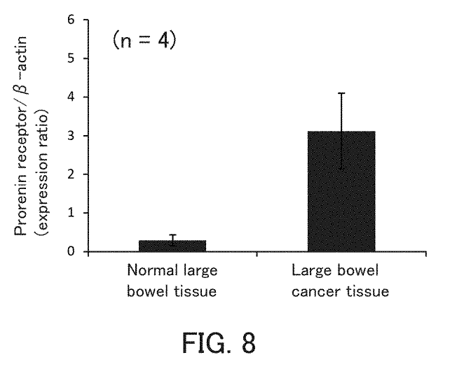

The results of the expression level of mRNA of the prorenin receptor gene in the large intestine cancer tissue are shown in FIG. 8. The horizontal axis indicates the types of test samples and the vertical axis indicates the relative expression level of mRNA of the prorenin receptor gene to mRNA of the β-actin gene. As can be seen from FIG. 8, the expression of mRNA of the prorenin receptor gene in the large intestine cancer tissues (n=4) was significantly higher than that in the normal large intestine tissues (n=4). From this result, it was found that there is a correlation between the occurrence of the large intestine cancer and the increase in the expression of mRNA of the prorenin receptor gene, which demonstrates that mRNA of the prorenin receptor gene in the large intestine tissue serves as a large intestine cancer marker.

### Example 6

The increase in the expression of the prorenin receptor protein in pancreatic cancer tissues was examined by immunohistochemical staining.

From twenty-two pancreatic cancer patients, normal epithelial tissues of the pancreatic duct, pancreatic cancer tissues, and epithelial tissues of the pancreatic duct containing preneoplastic lesion (PanIN) were collected. Paraffin sections were prepared with these tissues as test samples, and deparaffinization was performed using 100% xylene and 99.5% ethanol. Then, regarding each sample, endogenous peroxidase was inactivated using 100% ethanol and 30% hydrogen peroxide water. Next, after enclosing the section area of the test sample with a liquid blocker (water-repellent material), the sample was washed with phosphate buffer saline (PBS). Subsequently, the test sample was subjected to blocking at room temperature for 15 minutes using 10% normal goat serum (trade name: Histofine SAB-PO (R) kit, product of: NICHIREI BIOSCIENCES INC.).

After the blocking, the test sample was allowed to react at room temperature for 1 hour with 4000-fold diluted anti-rabbit (P) RR polyclonal antibody and then washed with PBS. Next, the test sample was allowed to react at room temperature for 30 minutes with Histofine Simple Stain MAX-PRO (MULTI) (product of: NICHIREI BIOSCIENCES INC.), which is a HRP-labeled secondary antibody. Subsequently, the test sample was washed with PBS and the color was developed with 3,3′-diaminobenzidine (DAB), which is a HRP coloring substrate. Then, the test sample was washed with running water and stained by haematoxylin and eosin (H&E) staining. The test sample after staining was washed with running water, dewatered with 99.5% ethanol and 100% xylene, and then immobilized. The test sample immobilized was sealed with marinol, and the expression site of the prorenin receptor stained brown with DAB was observed using an optical microscope.

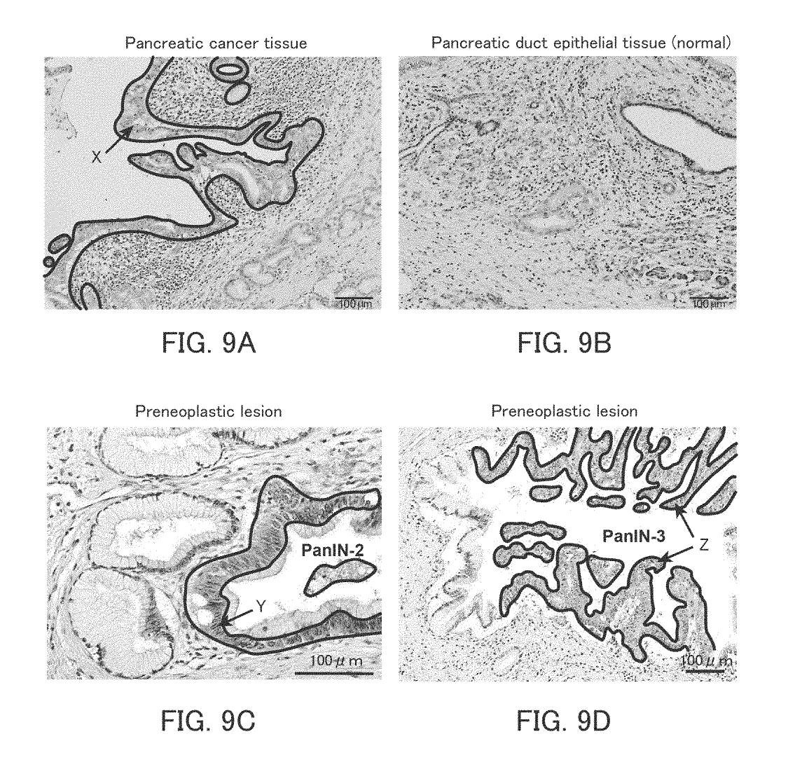

The results of the expression of the prorenin receptor protein in the pancreatic tissue are shown in FIGS. 9A to 9D. FIGS. 9A to 9D are tissue staining views each showing the expression of the prorenin receptor protein in the pancreatic tissue. FIG. 9A is a tissue staining view of the pancreatic cancer tissue, FIG. 9B is a tissue staining view of the normal epithelial tissue of the pancreatic duct, FIG. 9C is a tissue staining view of the epithelial tissue of the pancreatic duct containing PanIN-2, which is the middle stage of PanIN, and FIG. 9D is a tissue staining view of the epithelial tissue of the pancreatic duct containing PanIN-3, which is the late stage of PanIN. In FIGS. 9A to 9D, each of the scale bars has a scale unit of 100 μm, and the stained areas are enclosed by solid lines (for example, the areas indicated by arrows X, Y, and Z).

As can be seen from FIG. 9B, brown staining was hardly observed regarding the epithelial tissue of the pancreatic duct (normal). On the other hand, as can be seen from FIG. 9A, the area enclosed by the solid line (X) was stained brown in the pancreatic cancer tissue. Furthermore, as can be seen from FIGS. 9C and 9D, the respective areas enclosed by the solid lines (Y) and (Z) in the PanIN-2 and PanIN-3 were stained brown. That is, the expression of the prorenin receptor protein in the pancreatic cancer tissue and PanIN was higher than that in the normal epithelial tissue of the pancreatic duct. It is to be noted that although FIGS. 9A to 9D show tissue staining views of one pancreatic cancer patient, the expression of the prorenin receptor protein in the pancreatic cancer tissue and PanIN was increased in the same manner also in the tissues of other pancreatic cancer patients. From these results, it was found that there is a correlation of the formation of PanIN and the occurrence of the pancreatic cancer with the increase in the expression of the prorenin receptor protein, which demonstrates that the prorenin receptor protein in the pancreatic tissue serves as a marker showing high possibility of the pancreatic cancer occurring and a pancreatic cancer marker.

### Example 7

Human pancreatic cancer cell lines treated with siRNA (P) RR were transplanted to nude mice, and the suppression of growth of the pancreatic cancer cell lines and the decrease in the expression level of soluble prorenin receptor protein in the plasma in the nude mice were examined.

(1) Growth of Pancreatic Cancer Cell Line

siRNA (P) RR was transfected in the same manner as in Example 2 except that PANC-1, which is a human pancreatic cancer cell line, was used instead of PK-1, and PANC-1 (siRNA (P) RR group) transfected with siRNA was collected. Next, PANC-1 having 5×10⁵ cells was subcutaneously transplanted to the upper right flank of 5-week male BALB/c nude mouse (n=7) (product of CLEA Japan Inc.). Then, the major axis and minor axis of PANC-1-derived tumor in the upper right flank were measured using a micrometer (AS ONE corporation) 25 and 40 days after the transplantation. The tumor volume was calculated by the equation below. As a control, the tumor volume was measured in the same manner except that PANC-1 (scramble group) transfected with scrambled siRNA was used instead of siRNA (P) RR.

Cancer volume(mm³)=major axis×(minor axis)²×0.5 [Equation 1]

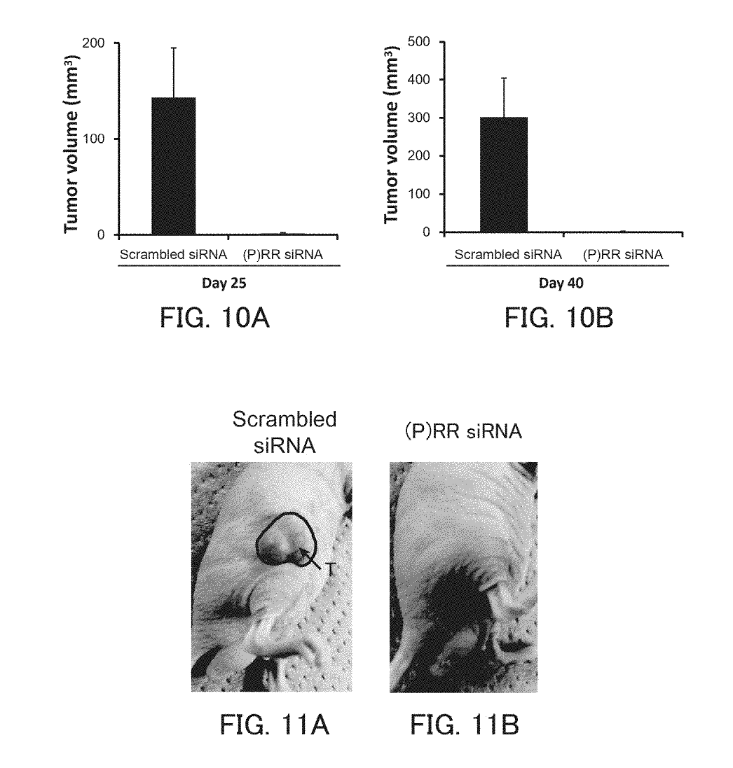

FIGS. 10A and 10B show the tumor volume and FIGS. 11A and 11B show the photographs of the mice 25 days after the transplantation. FIG. 10A is a graph showing the tumor volume 25 days after the transplantation and FIG. 10B is a graph showing the tumor volume 40 days after the transplantation. In FIGS. 10A and 10B, each of the horizontal axes indicates the types of siRNA and each of the vertical axes indicates the tumor volume. As can be seen from FIGS. 10A and 10B, the siRNA(P) RR group-derived tumor volume 25 and 40 days after the transplantation was significantly lower than that of the scramble group-derived tumor.

FIG. 11A is a photograph of the mouse 25 days after the scramble group transplantation and FIG. 11B is a photograph of the mouse 25 days after the si (P) RR group transplantation. As can be seen from FIGS. 11A and 11B, the formation of a tumor was observed in the area enclosed by the solid line (T) regarding the mouse 25 days after the scramble group transplantation. In contrast, no formation of a tumor was observed regarding the siRNA (P) RR group. From these results, it was found that the growth of the tumor in a biological body can be suppressed by suppressing the expression of the prorenin receptor.

(2) Expression Level of Prorenin Receptor Protein

Plasma was collected from the blood of the nude mouse 25 days after the transplantation. The expression level of the prorenin receptor protein in the plasma was measured in the same manner as in the item (1) in Example 3. Next, assuming that the expression level of the prorenin receptor in the plasma of the control was 1, the relative value of the expression level of the prorenin receptor in the plasma of the nude mouse transplanted with the siRNA (P) RR-transfected PANC-1 was obtained.

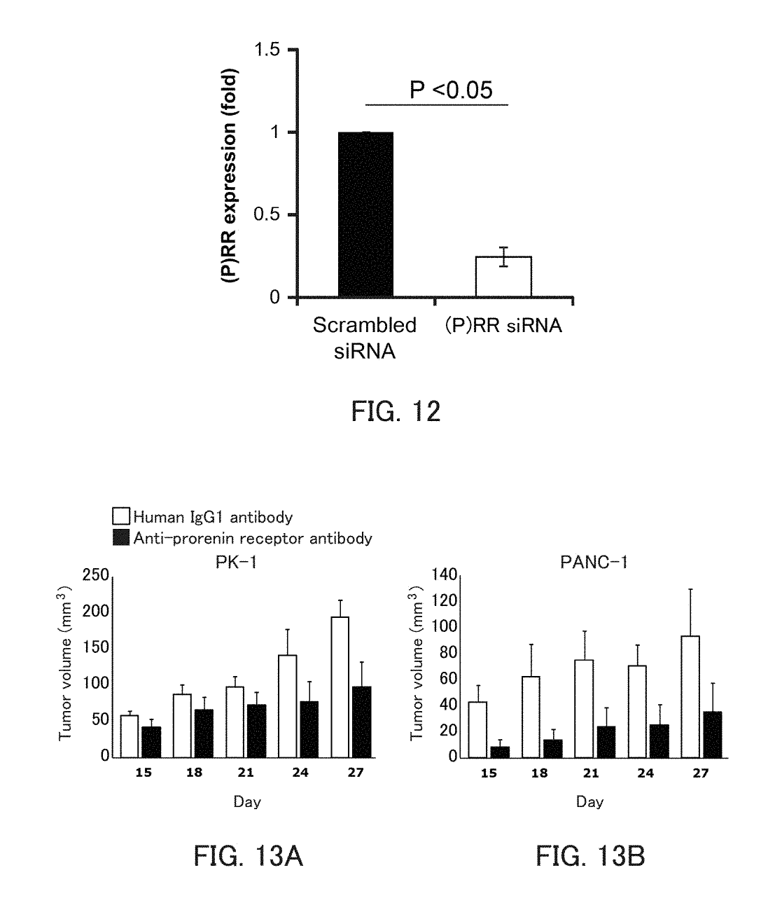

The results thereof are shown in FIG. 12. FIG. 12 is a graph showing the relative value of the expression level of the prorenin receptor in the plasma 25 days after the transplantation. In FIG. 12, the horizontal axis indicates the types of siRNA and the vertical axis indicates the relative expression level of the prorenin receptor. As can be seen from FIG. 12, the relative expression level of the prorenin receptor in the plasma of the nude mouse transplanted with the siRNA (P) RR group was significantly lower than that of the nude mouse transplanted with the scramble group. From these results, it was found that the expression level of the prorenin receptor in the plasma can be suppressed by suppressing the expression of the prorenin receptor of the tumor in the biological body.

### Example 8

Whether the growth of pancreatic cancer cells transplanted into nude mice can be suppressed by administrating an anti-prorenin receptor antibody to the mice was examined.

(1) Preparation of Anti-Prorenin Receptor Antibody

An antigen protein was prepared by binding a polypeptide (SEQ ID NO: 7) extending from the 200th to 213rd amino acid residues in the human prorenin receptor protein (SEQ ID NO: 6) to KLH (keyhole limpet hemocyanin). 150 to 200 μg of the antigen protein was administrated to a few tens of intradermal sites of a rabbit 7 to 10 times at two-week intervals, thus immunizing the rabbit (600 to 800 μg/kg body weight). The first immunization was performed using a mixture of the antigen protein and a complete adjuvant, and the second and subsequent immunizations were performed using a mixture of the antigen protein and an incomplete adjuvant. Next, after the increase in antibody titer against the polypeptide had been confirmed, whole blood was collected from the rabbit, and serum further was collected from the whole blood. The thus-collected serum was used as an antiserum. The concentration of a polyclonal anti-human prorenin receptor antibody (clone name: PRR1) in the antiserum was 0.6 μg/μ1. The homology (the alignment score between two different sequences) between the complete amino acid sequence of the human prorenin receptor protein used in the antigen protein and the complete amino acid sequence of a mouse prorenin receptor protein (SEQ ID NO: 10) was 92.29%. Thus, it can be said that the human prorenin receptor antibody contained in the antiserum also binds to the mouse prorenin receptor.

Mouse prorenin receptor protein
(SEQ ID NO: 10)
MAVLVVLLFFLVAGALGNEFSILRSPGSVVFRNGNWPIPGDRIPDVAALS
MGFSVKEDLSWPGLAVGNLFHRPRATIMVMVKGVDKLALPAGSVISYPLE
NAVPFSLDSVANSIHSLFSEETPVVLQLAPSEERVYMVGKANSVFEDLSV
TLRQLRNRLFQENSLLNSLPLNSLSRNNEVDLLFLSELQVLHDISSLLSR
HKHLAKDHSPDLYSLELAGLDELGKRYGEDSEQFRDASKILVDALQKFAD
DMYSLYGGNAVVELVTVKSFDTSLVRKSRTILEAKQENTQSPYNLAYKYN
LEYSVVFNLVLWIMIGLALAVIITSYNIWNMDPGYDSIIYRMTNQKIRID

(2) Suppression of Cancer Cell Growth In Vivo

One×10⁶ of PK-1 or PANC-1 cells, 30 μl of the antiserum (the antibody: 0.6 μg/μl) and 170 μl of PBS were mixed together. The resultant mixture (the whole amount) was transplanted into an upper right flank region of each of nude mice (n=10). At this time, the amounts of the cells and the antibody transplanted per kilogram body weight of each mouse were as follows: the cells: 50×10⁶; and the antibody: 0.9 mg. Next, after the transplantation, 10 μl of the antiserum (the antibody: 0.6 μg/μ1) was administrated into the PK-1- or PANC-1-derived tumor every three days. The amount of the antibody administrated in a single administration was 0.3 mg/kg body weight of the mouse. Then, on Days 15, 18, 21, 24, and 27 after the transplantation, the PK-1- or PANC-1-derived tumor volume in the upper right flank region was measured in the same manner as in Example 7. As a control, the tumor volume was measured in the same manner, except that, instead of the anti-prorenin receptor antibody, a human IgG1 antibody (WAKO) was administered in an amount of 1 mg/kg body weight of each mouse.

The results thereof are shown in FIGS. 13A and 13B. FIG. 13A is a graph showing the tumor volume in the case where PK-1 was transplanted, and FIG. 13B is a graph showing the tumor volume in the case where the PANC-1 was transplanted. In FIGS. 13A and 13B, the horizontal axis indicates the number of days elapsed after the transplantation, and the vertical axis indicates the tumor volume. In FIGS. 13A and 13B, filled bars indicate the results obtained regarding the anti-prorenin receptor administration group, and open bars indicate the results obtained regarding the human IgG1 antibody administration group. As can be seen from FIGS. 13A and 13B, in both the PK-1-transplanted nude mice and the PANC-1-transplanted nude mice, the tumor volumes on Days 15, 18, 21, 24, and 27 after the transplantation in the anti-prorenin receptor administration group were smaller than those in the human IgG1 antibody administration group. From these results, it was found that cancer growth can be suppressed by using an anti-prorenin receptor antibody in vivo.

### Example 9

The expression of a prorenin receptor protein in a human pancreatic duct epithelial cell line and in human pancreatic cancer cell lines was examined. The expression of the prorenin receptor protein in culture supernatants of these cell lines was also examined.

As the human pancreatic duct epithelial cell line, HPDE was used. As the human pancreatic cancer cell lines, PK-8, PCI-35, BxPC-3, PK-1, PANC-1, and MIAPaCa-2 were used. Each of the cell lines was cultured in a serum-free medium at 37° C. in 5% CO₂ for 24 hours. As the serum-free medium, RPMI-1640 (Sigma) was used. After the culture, the culture supernatant was collected. Next, 1 ml of the culture supernatant was concentrated using an Amicon Ultra-0.5 Centrifugal Filter Unit with Ultracel-10 membrane (Millipore), whereby a concentrate containing 20 μg of total protein was obtained. The concentrate was used as a culture supernatant sample.

After the collection of the culture supernatant, the cell line was lysed with a lysis buffer. The cell lysate was centrifuged at 12,500 rpm for 10 minutes, and then, the supernatant was collected. The thus-obtained supernatant was used as a cell lysate sample. Then, the expression of a prorenin receptor protein and a β-actin protein in the culture supernatant sample and the cell lysate sample was examined in the same manner as in the item (1) in Example 3.

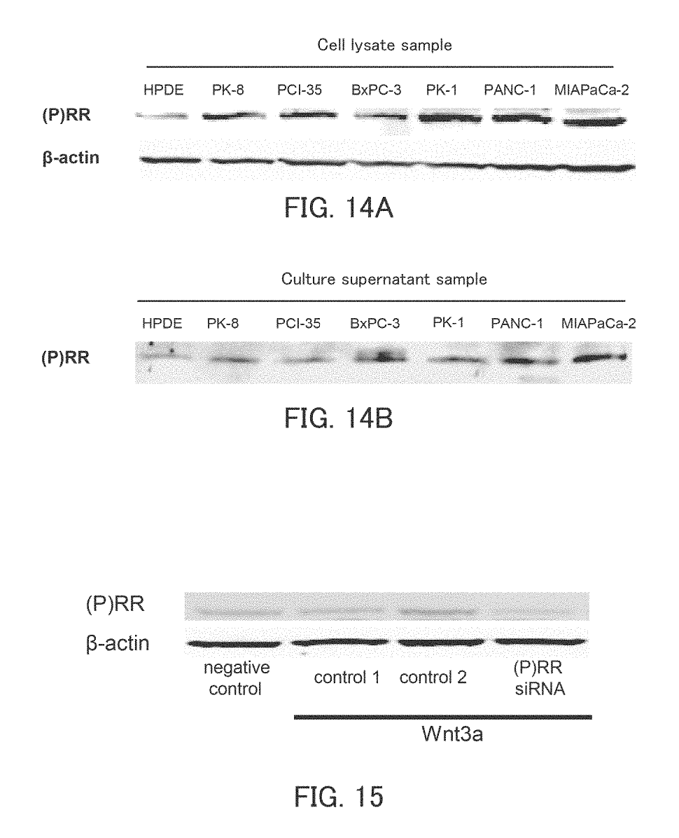

The results thereof are shown in FIGS. 14A and 14B. FIG. 14A is a Western Blot photograph showing the expression of the prorenin receptor protein in the cell lysate samples of the respective cell lines. FIG. 14B is a Western Blot photograph showing the expression of the prorenin receptor protein in the culture supernatant samples of the respective cell lines. In FIGS. 14A and 14B, the cell lines used are indicated above the photograph, and the detected proteins are indicated on the left of the photograph. As can be seen from FIG. 14A, the expression of the prorenin receptor protein was observed in the cell lysate samples of all the cell lines. Also, as can be seen from FIG. 14B, the expression of the prorenin receptor protein was observed in the culture supernatant samples of all the cell lines. These results confirm the expression of the prorenin receptor protein in the human pancreatic duct epithelial cell line and the human pancreatic cancer cell lines, and also, in the culture supernatants of these cell lines.

### Example 10

Whether Wnt signals are suppressed by suppressing the expression of a prorenin receptor was examined.

(1) Expression of Phosphorylated LRP6

In the same manner as in Example 2, siRNA (P) RR was transfected into PK-1 (siRNA (P) RR group). After the transfection, a recombinant human Wnt3a was added so that the concentration thereof became 150 ng/ml. Ten minutes after the addition of the recombinant human Wnt3a, PK-1 was collected and lysed with a lysis buffer. Thus, a test sample was prepared. Next, the expression of a prorenin receptor protein and a β-actin protein was examined in the same manner as in the item (1) in Example 3.

The expression level of a phosphorylated LRP6 protein or LRP6 protein in the above test sample was measured in the same manner as in the item (1) in Example 3, except that an anti-phosphorylated LRP6 antibody (Ser 1490, Cell Signaling Technology) or an anti-LRP6 antibody (clone name: C47E12, Cell Signaling Technology) was used instead of the anti-prorenin receptor antibody. Also, the expression levels of the phosphorylated LRP6 protein and the LRP6 protein were measured in the same manner, except that: as a negative control, the siRNA was not transfected and the Wnt3a was not added; as control 1, the siRNA was not transfected; and as control 2, a scrambled siRNA was transfected instead of the siRNA (P) RR.

Furthermore, regarding each sample, the ratio of the expression level of the phosphorylated LRP6 protein to the expression level of the LRP6 protein was determined (this ratio is referred to as the “phosphorylated LRP6 expression level ratio”). Then, assuming that the phosphorylated LRP6 expression level ratio in the negative control was 1, the relative value of the phosphorylated LRP6 expression level ratio in each sample was calculated.

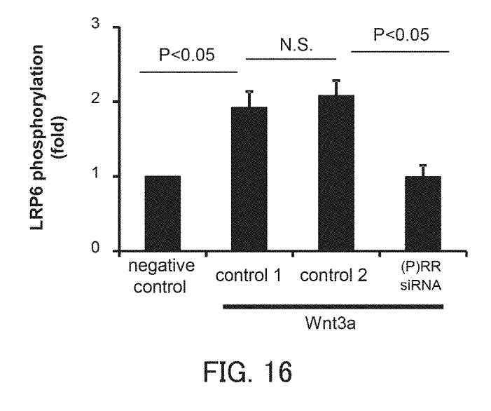

FIG. 15 shows the expression of the prorenin receptor protein and the β-actin protein. FIG. 16 shows the relative values of the phosphorylated LRP6 expression level ratio. FIG. 15 is a Western Blot photograph showing the expression of the prorenin receptor protein and the β-actin protein. In FIG. 15, the siRNA used for the treatment and the presence or absence of the Wnt3a stimulation are indicated below the photograph, and the detected proteins are shown on the left of the photograph. As can be seen from FIG. 15, the expression level of the prorenin receptor protein in the siRNA (P) RR group was lower than those in the negative control and the controls 1 and 2.

FIG. 16 is a graph showing the relative values of the phosphorylated LRP6 expression level ratio. In FIG. 16, the horizontal axis indicates the siRNA used for the treatment and the presence or absence of the Wnt3a stimulation, and the vertical axis indicates the relative value of the phosphorylated LRP6 expression level ratio. As can be seen from FIG. 16, the relative value of the phosphorylated LRP6 expression level ratio after the Wnt3a stimulation in the siRNA (P) RR group was lower than those in the negative control and the controls 1 and 2. From these results, it was confirmed that Wnt signals are suppressed by suppressing the expression of a prorenin receptor protein.

(2) Expression of Active β-Catenin and Cyclin D1

A test sample was prepared in the same manner as in the above item (1). Next, the expression levels of an active β-catenin protein and a Cyclin D1 protein in the test sample were determined in the same manner as in the item (1) in Example 3, except that an anti-active β-catenin antibody (anti-ABC, Millipore) or an anti-Cyclin D1 antibody was used instead of the anti-prorenin receptor antibody. The expression levels of the active β-catenin protein and the Cyclin D1 protein also were determined in the same manner, except that: as a negative control, a scrambled siRNA was transfected instead of the siRNA (P) RR and the Wnt3a was not added; and as a control, a scrambled siRNA was transfected instead of the siRNA (P) RR. Then, assuming that the expression levels of the active β-catenin protein and the Cyclin D1 protein in the negative control were 1, the relative values of the expression levels of the active β-catenin protein and the Cyclin D1 protein in each sample were determined.

Also, regarding the BxPC-3 and the PANC-1, the relative values of the expression levels of the active β-catenin protein and the Cyclin D1 protein were determined in the same manner.

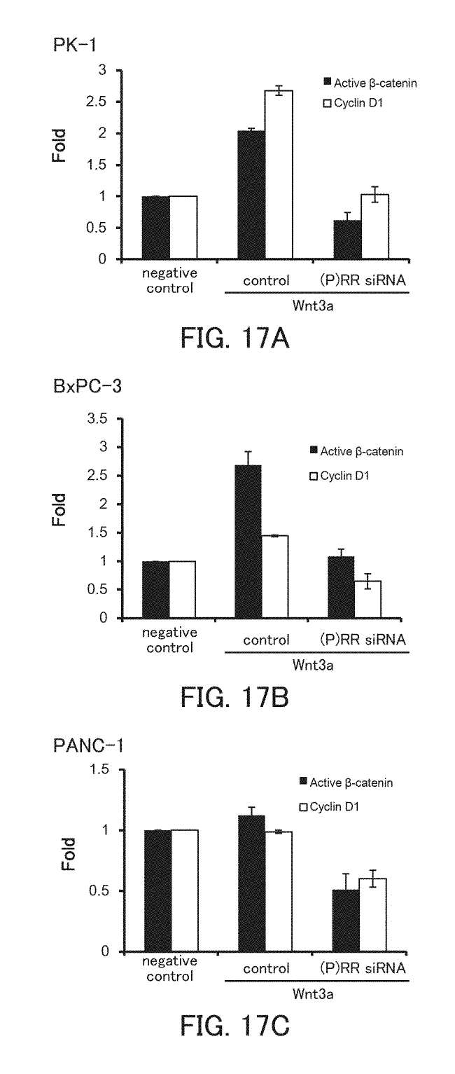

The results thereof are shown in FIGS. 17A to 17C. FIGS. 17A to 17C are graphs each showing the relative values of the expression levels of the active β-catenin protein and the Cyclin D1 protein. FIG. 17A shows the results obtained when the PK-1 was used. FIG. 17B shows the results obtained when the BxPC-3 was used. FIG. 17C shows the results obtained when the PANC-1 was used. In FIGS. 17A to 17C, the horizontal axis indicates the siRNA used for the treatment and the presence or absence of the Wnt3a stimulation, and the vertical axis indicates the relative expression level. In FIGS. 17A to 17C, filled bars indicate the relative expression levels of the active β-catenin protein, and open bars indicate the relative expression levels of the Cyclin D1 protein.

As can be seen from FIGS. 17A to 17C, the relative expression levels of the active β-catenin protein and the Cyclin D1 protein in the siRNA (P) RR group were significantly lower than those in the control, and were nearly equal to or smaller than those in the negative control. The active β-catenin protein and the Cyclin D1 protein are induced by Wnt signals. Thus, from these results, it was confirmed that Wnt signals are suppressed by suppressing the expression of a prorenin receptor protein.

### Example 11

PK-1 was seeded into a 6-well plate at a density of 5×10⁴ cells/well. Next, siRNA (P) RR was transfected into the PK-1 in the same manner as in Example 2 (siRNA (P) RR group). After the transfection, a recombinant human Wnt3a was added so that the concentration thereof became 150 ng/ml, and the resultant culture was incubated for 48 hours. After the culture, 100 μl of a water-soluble tetrazolium salt (WST-1) reagent further was added to each well, and the resultant culture was incubated for 2 hours. Then, using a plate reader, the absorbance at 450 nm in each well was measured to determine the proliferation potency. The proliferation potency was determined in the same manner, except that: as a negative control, the siRNA was not transfected and the Wnt3a was not added; as control 1, the siRNA was not transfected; and as control 2, a scrambled siRNA was transfected instead of the siRNA (P) RR.

Then, assuming that the proliferation potency in the negative control was 1, the relative value of the proliferation potency in each sample was determined. Furthermore, regarding each of the BxPC-3 and the PANC-1, the relative value of the proliferation potency was determined in the same manner.

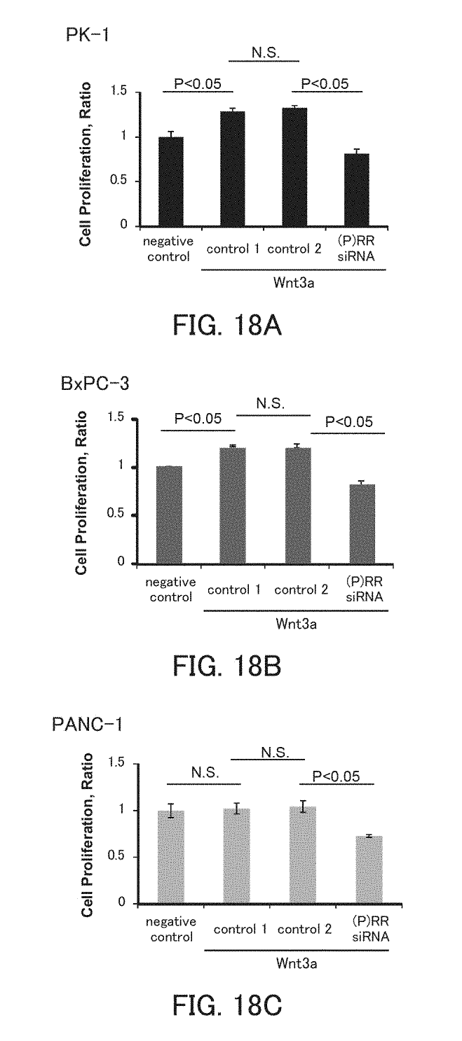

The results thereof are shown in are shown in FIGS. 18A to 18C. FIGS. 18A to 18C are graphs each showing the relative value of the proliferation potency. FIG. 18A shows the results obtained when the PK-1 was used. FIG. 18B shows the results obtained when the BxPC-3 was used. FIG. 18C shows the results obtained when the PANC-1 was used. In FIGS. 18A to 18C, the horizontal axis indicates the siRNA used for the treatment and the presence or absence of the Wnt3a stimulation, and the vertical axis indicates the relative proliferation potency.

As can be seen from FIGS. 18A to 18C, when any of the human pancreatic cancer cell lines was used, the relative proliferation potency in the siRNA (P) RR group was lower than those in the negative control and the controls 1 and 2. From these results, it was confirmed that the proliferation of human pancreatic cancer cell lines is suppressed by suppressing the expression of a prorenin receptor protein.

### Example 12

Whether apoptosis is induced by suppressing the expression of a prorenin receptor was examined.

(1) Measurement of Apoptosis Cells

In the same manner as in Example 2, siRNA (P) RR was transfected into PK-1 (siRNA (P) RR group). After the transfection, a recombinant human Wnt3a was added so that the concentration thereof became 150 ng/ml, and the resultant culture was incubated for 48 hours. Next, the cultured PK-1 was centrifuged and collected. The collected PK-1 was suspended in 0.5 ml of ice-cooled 70% ethanol, and the thus-obtained suspension was stored at −20° C. for 24 hours. After the storage, the PK-1 was washed with PBS, and then suspended in 10 mg/ml RNase A (Macherey-Nagel)-containing PBS. The thus-obtained suspension was incubated at 37° C. for 30 minutes. Thereafter, the suspension was subjected to staining with 1 mg/ml PI (Sigma-Aldrich)-containing PBS at 37° C. for 30 minutes.

The stained PK-1 was washed with PBS. Then, the PK-1 was suspended in PBS again. Forward-scattered light (FS), side-scattered light (SS), and the DNA content in this suspension were measured using a DNA flow cytometer (Beckman Coulter, Inc.). Also, on the basis of the DNA content, the proportion of cells in each cell cycle was calculated. As a control, forward-scattered light (FS), side-scattered light (SS), and the DNA content were measured and the proportion of cells in each cell cycle was calculated in the same manner, except that a scrambled siRNA was transfected instead of the siRNA (P) RR. Furthermore, regarding PANC-1, the proportion of cells in each cell cycle was calculated in the same manner.

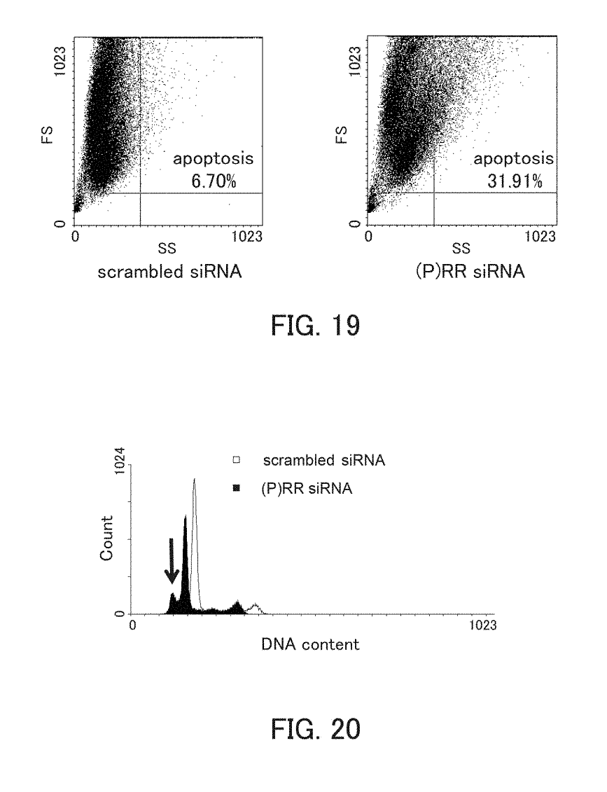

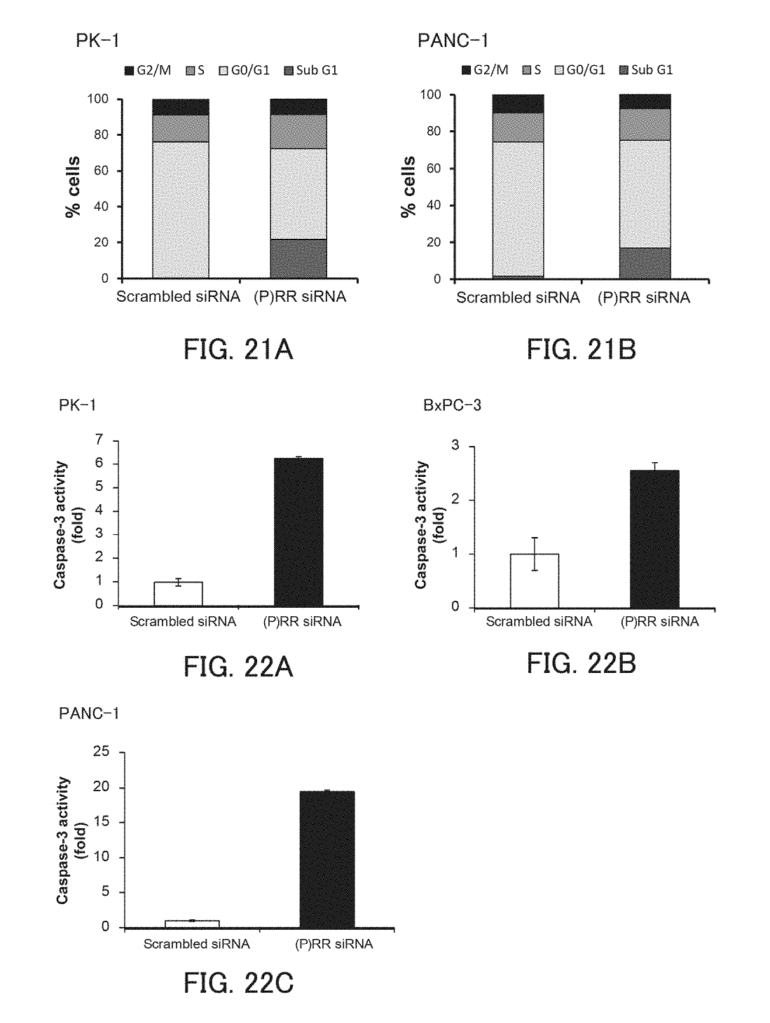

FIG. 19 shows dot plots each showing the FS and the SS. FIG. 20 is a histogram showing the DNA content. FIGS. 21A and 21B are graphs each showing the proportion of cells in each cell cycle. The dot plots shown in FIG. 19 show the proportion of apoptosis cells. In FIG. 19, the horizontal axis indicates the SS, and the vertical axis indicates the FS, and the number shown inside each dot plot indicates the proportion of apoptosis cells. As can be seen from FIG. 19, in the siRNA (P) RR group, the proportion of apoptosis cells, which are cells included in the upper right quarter of the dot plot, was greater than that in the control.

FIG. 20 is a histogram showing the DNA content. In FIG. 20, the horizontal axis indicates the fluorescence intensity of the PI, and the vertical axis indicates the count. Also, in FIG. 20, the filled histogram indicates the result obtained regarding the siRNA (P) RR group, and the open histogram indicates the result obtained regarding the control. As can be seen from FIG. 20, in the siRNA (P) RR group, apoptosis cells having a relatively low DNA content as indicated with the arrow were observed. In contrast, in the control, apoptosis cells were not observed.

FIGS. 21A and 21B are graphs each showing the proportion of cells in each cell cycle. FIG. 21A shows the results obtained when the PK-1 was used. FIG. 21B shows the results obtained when the PANC-1 was used. In FIGS. 21A and 21B, the horizontal axis indicates the siRNA used for the treatment, and the vertical axis indicates the proportion of cells in each cell cycle.

As can be seen from FIGS. 21A and 21B, when any of the human pancreatic cancer cell lines was used, the proportion of SubG1 group cells, which are apoptosis cells, in the siRNA (P) RR group was greater than that in the control. From these results, it was confirmed that apoptosis of human pancreatic cancer cell lines is induced by suppressing the expression of a prorenin receptor protein.

(2) Measurement of Activation of Caspase 3

PK-1 was cultured and collected in the same manner as in the above item (1). The activity of caspase 3 was measured using an APOPCYTE (Kit name: Caspase-3 Colorimetric Assay Kit, Medical & Biological laboratories) in accordance with its protocol. As a control, the activity of caspase 3 was measured in the same manner, except that a scrambled siRNA was transfected instead of the siRNA (P) RR. Also, regarding the BxPC-3 and the PANC-1, the activity of the caspase 3 was measured in the same manner.

The results thereof are shown in FIGS. 22A to 22C. FIGS. 22A to 22C are graphs each showing the activity of the caspase 3. FIG. 22A shows the results obtained when the PK-1 was used. FIG. 22B shows the results obtained when the BxPC-3 was used. FIG. 22C shows the result obtained when the PANC-1 was used. In FIGS. 22A to 22C, the horizontal axis indicates the siRNA used for the treatment, and the vertical axis indicates the activity of the caspase 3. As can be seen from FIGS. 22A to 22C, when any of the human pancreatic cancer cell lines was used, the activity of the caspase 3 inducing apoptosis in the siRNA (P) RR group was higher than that in the control. From these results, it was confirmed that caspase 3, which induces apoptosis, is activated by suppressing the expression of a prorenin receptor protein.

### Example 13

The increase in the concentration of the prorenin receptor in the plasma of brain tumor patients was examined.

Plasma was collected from blood of each of the following subjects: healthy males (n=4), healthy females (n=2), male brain tumor patients (n=5), and female brain tumor patients (n=10). The concentration of the prorenin receptor in the plasma of each subject was measured in the same manner as in Example 1.

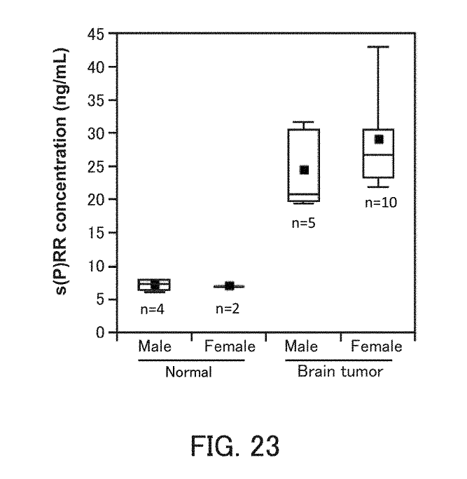

The results thereof are shown in FIG. 23. FIG. 23 is a graph showing the concentration of the prorenin receptor in the plasma. In FIG. 23, the horizontal axis indicates the type of the test sample, and the vertical axis indicates the concentration of the prorenin receptor. As can be seen from FIG. 23, in both the male and female subjects, the concentrations of the prorenin receptor in the plasma of the brain tumor patients (Brain tumor) were significantly higher than those in the plasma of the healthy subjects (Normal). From these results, it was found that there is a correlation between the occurrence of the brain tumor and the increase in the concentration of the prorenin receptor in the plasma regardless of the sex of a subject, which demonstrates that the prorenin receptor in plasma serves as a brain tumor marker.

While the invention has been described with reference to the embodiments and the examples, the invention is not limited to these embodiments and examples. Various changes which are understood by those skilled in the art in the scope of the present invention can be applied to the configuration and detail of the present invention.

This application is based upon and claims the benefit of priority from Japanese patent application No. 2012-199508, filed on Sep. 11, 2012, the disclosure of which is incorporated herein its entirety by reference.

### INDUSTRIAL APPLICABILITY

As described above, the present invention allows a morbidity risk of a cancer to be tested by measuring a prorenin receptor expression level. Moreover, a prorenin receptor can be a target of a cancer. Thus, the screening method according to the present invention allows a candidate substance for use in a cancer therapy to be obtained using a prorenin receptor as a target. Therefore, the cancer marker according to the present invention is a useful marker in a clinical field and a biochemical field.

### SEQUENCE LISTING

T13031WO_2013.09.06_ST25.txt

1. A test and therapeutic method, comprising:
measuring a prorenin receptor expression level in a biological specimen obtained from a subject, wherein
the biological specimen is a blood specimen, and
a cancer to be tested is at least one of a pancreatic cancer and a brain tumor;
comparing the prorenin receptor expression level in the biological specimen obtained from the subject with a reference value to test the morbidity risk of the cancer in the subject, wherein
the reference value is a prorenin receptor expression level in a biological specimen obtained from a healthy subject or a cancer patient, and
when the prorenin receptor expression level in the biological specimen obtained from the subject is higher than that in the biological specimen obtained from the healthy subject, or identical to or higher than that in the biological specimen obtained from the cancer patient, determining the subject to have a morbidity risk from the pancreatic cancer or brain tumor; and
administering a therapeutic composition for the pancreatic cancer or brain tumor to the subject determined to have the high morbidity risk from the pancreatic cancer or brain tumor, the therapeutic composition comprising at least one of a binding substance for binding to a prorenin receptor and an expression suppressive substance for suppressing a prorenin receptor expression.

2. The test and therapeutic method according to claim 1, wherein
the cancer is a pancreatic cancer.

3. The test and therapeutic method according to claim 1, wherein
the brain tumor is at least one selected from the group consisting of glioma, astrocytoma, primary central nervous system malignant lymphoma, and cavernous hemangioma.

4-5. (canceled)

6. The test and therapeutic method according to claim 1, wherein
the prorenin receptor expression level is at least one of an expression level of mRNA of a prorenin receptor and an expression level of a prorenin receptor protein.

7-26. (canceled)

27. The test and therapeutic method according to claim 1, wherein
the binding substance for binding to a prorenin receptor comprises an antibody to a prorenin receptor or an antigen binding fragment of the antibody.

28. The test and therapeutic method according to claim 27, wherein
the antibody or the antigen binding fragment binds a region extending from the 200th to 213rd amino acid residues in an amino acid sequence (SEQ ID NO: 6) of a human prorenin receptor protein.

29. An antibody to a prorenin receptor or an antigen binding fragment of the antibody, wherein
the antibody or the antigen binding fragment binds a region extending from the 200th to 213rd amino acid residues in an amino acid sequence (SEQ ID NO: 6) of a human prorenin receptor protein.

30. A method for treating a pancreatic cancer, comprising the step of administrating an antibody to a prorenin receptor or an antigen binding fragment of the antibody to a subject.

31. The method for treating a pancreatic cancer according to claim 30, wherein
the antibody or the antigen binding fragment binds a region extending from the 200th to 213rd amino acid residues in an amino acid sequence (SEQ ID NO: 6) of a human prorenin receptor protein.

32. The method for treating a pancreatic cancer according to claim 30, wherein
the antibody or the antigen binding fragment is administrated in a pancreatic cancer.
